(19) United States
(12) Patent Application Publication
(10) Pub. No.: US 2021/0290808 A1
(43) Pub. Date: Sep. 23, 2021

(54) DRYER AND SANITIZER FOR RECHARGEABLE ELECTRONIC DEVICES

(71) Applicant: Ear Technology Corporation, Johnson City, TN (US)

(72) Inventors: Daniel R. Schumaier, Elizabethton, TN (US); Karlee D. Harrison, Johnson City, TN (US)

(73) Assignee: Ear Technology Corporation, Johnson City, TN (US)

(21) Appl. No.: 17/339,055

(22) Filed: Jun. 4, 2021

Related U.S. Application Data
(63) Continuation-in-part of application No. 16/742,156, filed on Jan. 14, 2020, now abandoned.
Application No. 16/742,156 is a continuation-in-part of application No. 16/665,045, filed on Oct. 28, 2019.
(60) Provisional application No. 62/883,761, filed on Aug. 7, 2019.

(51) Int. Cl. A61L 2/26; F26B 3/04; A61L 2/10

(52) U.S. Cl. CPC A61L 2/26; F26B 3/04; A61L 2202/14; A61L 2202/11; A61L 2202/16; A61L 2/10

(57) ABSTRACT

A dryer and sanitizer unit includes a drying chamber for enclosing one or more portable rechargeable electronic devices. The drying chamber is defined by a base and a repositionable lid that covers the base. The base includes a substantially horizontal surface upon which the electronic devices are disposed. Air vents are disposed around a perimeter of the horizontal surface through which warm air may flow into the drying chamber to contact the electronic devices. The air vents at least partially surround the electronic devices disposed on the base. The unit also includes one or more disinfecting light sources, such as UV-C light sources, disposed within the drying chamber.

### CROSS REFERENCES TO RELATED APPLICATIONS

This application claims priority as a continuation-in-part of U.S. patent application Ser. No. 16/665,045, filed Oct. 28, 2019, and U.S. patent application Ser. No. 16/742,156, filed Jan. 14, 2020, both entitled DRYER AND SANITIZER FOR RECHARGEABLE ELECTRONIC DEVICES, both of which claim priority to U.S. Provisional Application Ser. No. 62/883,761, entitled DRYER AND SANITIZER FOR RECHARGEABLE ELECTRONIC DEVICES, filed Aug. 7, 2019, the entire disclosures of which are incorporated by reference herein.

### FIELD

This disclosure relates to drying and sanitizing electronic devices. More particularly, this disclosure relates to a dryer and sanitizer unit configured for drying and sanitizing a wide variety of rechargeable electronic devices including, but not limited to, cell phones, hearing aids, watches, earphones, headphones, and their chargers, which come in many different sizes and shapes from different manufacturers.

### BACKGROUND

Improvement is desired in devices for charging, drying and sanitizing electronic devices. During use, the batteries of electronic devices such as cell phones, hearing aids, earphones, headphones, watches, and the like discharge, and such devices may collect bacteria and gather moisture during use that can be harmful to the electronic devices.

What is desired is a convenient way to recharge the electronic devices while also sanitizing and drying the electronic devices at the same time.

The present disclosure advantageously provides an easy-to-use unit that enables the charging of electronic devices while the electronic devices are sanitized and dried.

### SUMMARY

The above and other needs are met by a dryer and sanitizer unit that includes a drying chamber for enclosing one or more portable rechargeable electronic devices. The drying chamber is defined by a base and a repositionable lid that covers the base. The base includes a substantially horizontal surface upon which the one or more electronic devices are disposed. Air vents are disposed around a perimeter of the horizontal surface through which warm air may flow into the drying chamber to contact the one or more electronic devices. The air vents at least partially surround the one or more electronic devices disposed on the base. In a preferred embodiment, one or more disinfecting light sources, such as UV-C light sources, are disposed within the drying chamber.

In some embodiments, the lid or the base includes one or more passages through which electrical cords associated with the one or more portable rechargeable electronic devices may pass out of the drying chamber.

In some embodiments, the unit includes a thermostat for measuring air temperature in the drying chamber.

In some embodiments, the unit includes a microprocessor operably associated with the thermostat for controlling the air temperature in the drying chamber to maintain the air temperature above a predetermined drying temperature and below a predetermined upper temperature corresponding to a temperature above which rechargeable batteries of the portable rechargeable electronic devices are vulnerable to overheating.

In some embodiments, the one or more disinfecting light sources are disposed in the base.

In some embodiments, the unit includes a fan and a heater in flow communication with the air vents to cause warm air to flow through the air vents into the drying chamber.

In some embodiments, the unit includes air vents disposed in the lid to promote flow of the warm air over the portable rechargeable electronic devices and to provide a path for the warm air to exit the drying chamber.

In some embodiments, the disinfecting light sources are operated for a predetermined time sufficient to disinfect the portable rechargeable electronic devices, after which the disinfecting light sources are turned off and the heater and fan are operated for a predetermined drying cycle.

In some embodiments, the lid has a square pyramidal shape with a flat top.

In some embodiments, an interior surface of the lid is UV-reflective.

In some embodiments, the unit includes a lid open detection circuit that is operably associated with both the lid and the one or more disinfecting light sources. The lid open detection circuit is operable to turn off the one or more disinfecting light sources if the lid is opened while the one or more disinfecting light sources are turned on.

In some embodiments, the unit includes a wireless nearfield proximity charger integrated into the base.

### BRIEF DESCRIPTION OF THE DRAWINGS

Further advantages of the disclosure are apparent by reference to the detailed description when considered in conjunction with the figures, which are not to scale so as to more clearly show the details, wherein like reference numbers indicate like elements throughout the several views, and wherein:

FIG. 1 is a perspective view of first embodiment of a dryer and sanitizer unit according to the disclosure as used to dry and sanitize an electronic device such as a hearing aid;

FIG. 2 depicts the dryer and sanitizer unit of FIG. 1 with a charger for a hearing aid installed;

FIG. 3 depicts the dryer and sanitizer unit of FIG. 1 with a charger for a hearing aid and a hearing aid installed on the charger;

FIGS. 4-7 depict use of the dryer and sanitizer unit of FIG. 1 to dry and sanitize one or more hearing aids;

FIG. 8 depicts a functional block diagram of electrical components of embodiments of the dryer and sanitizer unit;

FIG. 9 depicts a schematic diagram of an electrical circuit embodiments of the dryer and sanitizer unit;

FIG. 10 is a perspective view of a second embodiment of a dryer and sanitizer unit according to the disclosure;

FIG. 11 is a perspective view of the dryer and sanitizer unit of FIG. 10 with the lid open and a cell phone and a charger for a cell phone installed;

FIG. 12 is a perspective view of the dryer and sanitizer unit of FIG. 10 with the lid open and a cell phone disposed on the base and attached to a charging cable;

FIG. 13 is a rear elevation view of the dryer and sanitizer unit of FIG. 10 showing an opening for a power cord and power connections to the unit;

FIG. 14 is a perspective view of a dryer and sanitizer unit according the second embodiment of the disclosure with the lid open to show a built-in wireless nearfield proximity charger in the base;

FIG. 15 is a cross-sectional view (not to scale) of the dryer and sanitizer unit according to FIG. 10; and

FIG. 16 is a perspective view showing the flow of air through the dryer and sanitizer unit according to the second embodiment of the disclosure.

### DETAILED DESCRIPTION

### First Embodiment

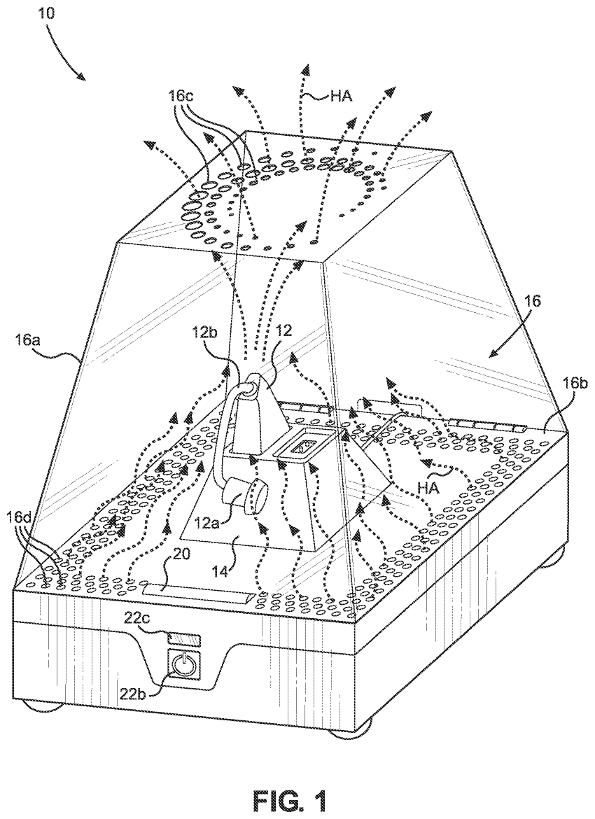

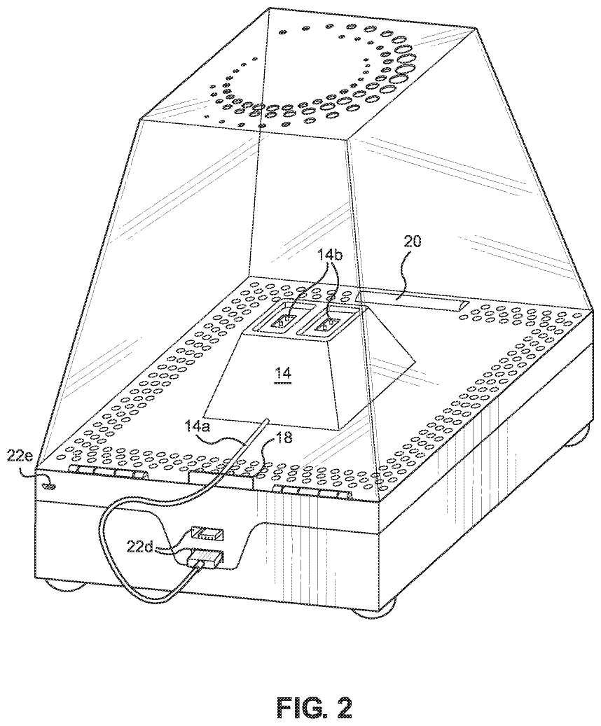

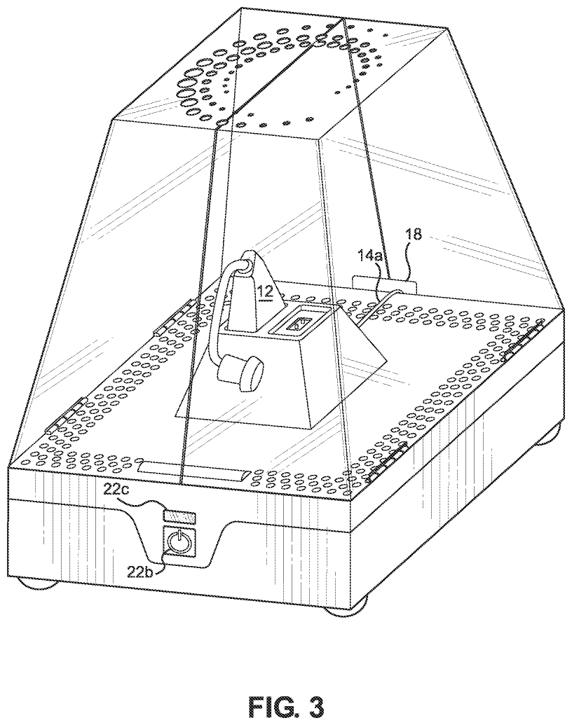

With initial reference to FIGS. 1-3 there is shown a first embodiment of a dryer and sanitizer unit 10 according to the disclosure configured for drying and sanitizing rechargeable electronic devices, such as hearing instruments in the nature of a hearing aid 12, including tips 12a and tubes 12b and seated in a charger 14 having an electric cord 14a. The hearing aid 12 may be a conventional hearing aid, and the charger 14 may be specifically compatible with the hearing aid 12. The charger 14 may include a pair of charging receptacles 14b for receiving a pair of the hearing aids 12.

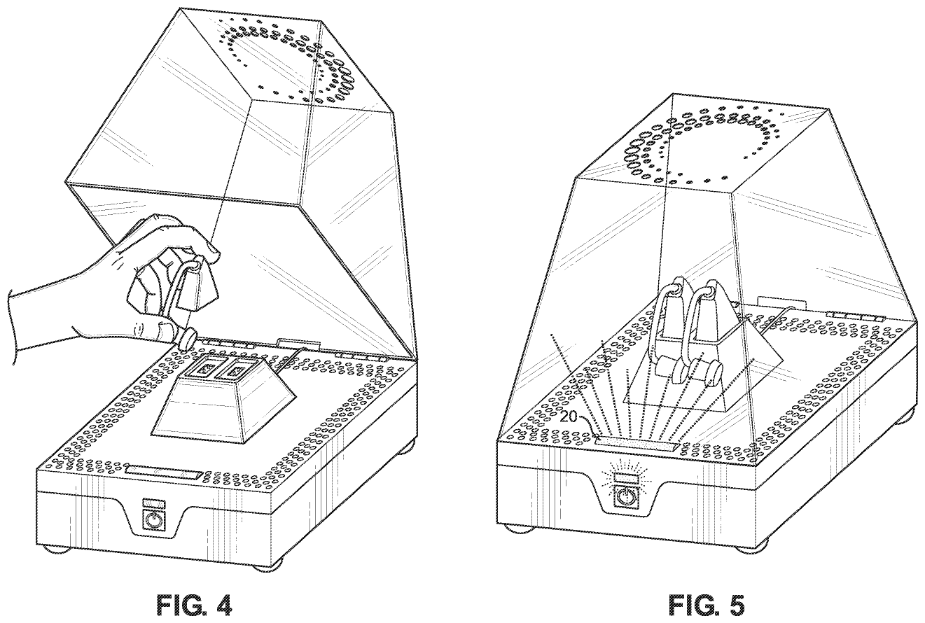

FIGS. 4-7 depict operation of the unit 10 to charge, dry, and sanitize rechargeable devices, such as a pair of the hearing aids 12. In overview, the unit 10 is configured to receive the hearing aids 12 on the charger 14 to enable their charging, drying and sanitization. In this regard, initially the hearing aids are disinfected by application of sanitizing illumination. Following or simultaneous with the sanitization, heated dry air is flowed over the hearing aids to remove moisture.

The dryer and sanitizer unit 10 is configured to include a drying chamber 16 defined by a lid 16a over a base 16b. The unit 10 also includes one or more passages 18 for passage of electric cords, such as the cord 14a, out of the drying chamber. The passages 18 are desirably located on the lid 16a but could be provided on the base 16b if desired.

Preferred embodiments of the unit 10 also include one or more disinfecting light sources 20 located within the drying chamber 16. The disinfecting light sources 20 are preferably UV-C lamps such as high intensity 50 mm linear germicidal lamps operating at a wavelength of 253.7 nm and rated at 70 uW/cm².

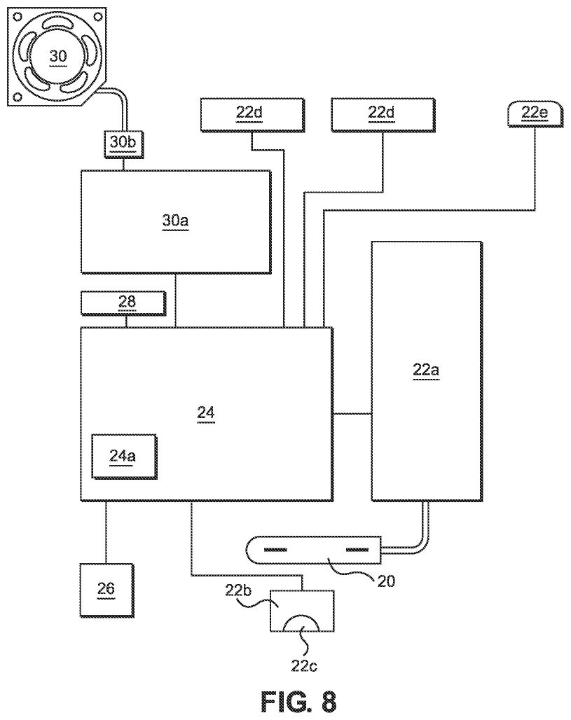

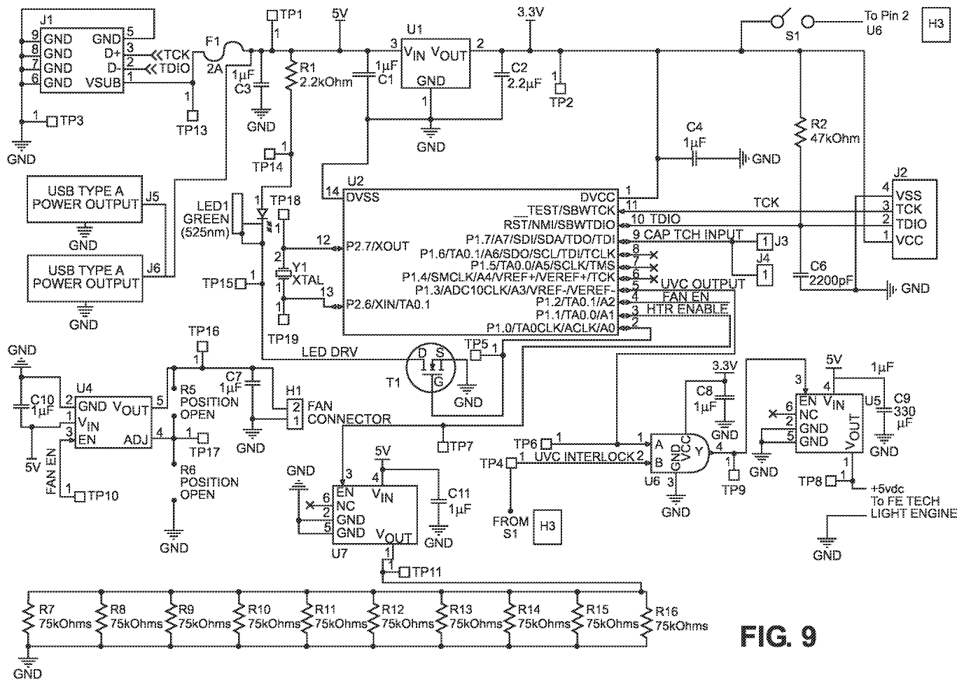

With continued reference to FIG. 8, various electronic devices of an embodiment of the unit 10 are shown as located in the base 16b. The light sources 20 are preferably in electrical communication with a lamp control circuit 22a. Electrical power is controlled by an on/off switch 22b, such as a capacitive touch switch, with an LED indicator 22c to indicate that the unit 10 is on. Electrical power may be supplied to the charger 14 by plugging the cord 14a into one or more power outlets 22d to which power is supplied under control of a microprocessor 24. The unit 10 preferably receives 5-volt direct current electrical power via one or more input power connectors 22e, such as USB micro connectors.

The lamp control circuit 22a may be programmed to operate the light sources 20 for a predetermined time sufficient to provide a 4-log theoretical kill rate of 99.99%. A desired operation time of the light sources 20 is from about 1.5 to about 3 minutes or longer to achieve this level of disinfection. In this regard, the lamp control circuit 22a is also in electrical communication with the microprocessor 24, which is configured to control the operation of the lamp control circuit 22a. To enhance the effect of the light sources 20, one or more interior surfaces of the lid 16a may be UV-reflective.

In some preferred embodiments, the microprocessor 24 is also operably associated with a lid open detection circuit 26 that is in communication with both the lid 16a and the light sources 20. The lid open detection circuit 26 is operable to turn off the light sources 20 if the lid 16a is opened during operation of the disinfecting light sources 20.

The lid 16a preferably has a square pyramidal shape with a flat top that includes air outlet vents 16c. The base 16b includes air inlet vents 16d through which heated dry air HA is supplied into the drying chamber 16. In one embodiment, the lid 16a is formed by two independently-hinged halves that operate like a clam shell.

The heated dry air HA is preferably supplied at a temperature that fits the specification of the manufacturer of the batteries for the hearing aids. Generally speaking, however, it has been observed that damage can occur to some rechargeable batteries for hearing aids if they are exposed to temperatures above about 113 degrees F. (45 degrees C.) for prolonged periods of time. The flow rate and flow time of the heated dry air HA is selected based on observation of parameters that provide optimal drying, and the microprocessor 24 is programmed to provide a desired air temperature as sensed by a thermostat 24a, for a predetermined time at a predetermined fan speed to provide the desired drying effect.

A heater 28 is also operably associated with the microprocessor 24. In addition, the microprocessor 24 is operably associated with a fan 30 connected to a fan circuit 30a via a fan connector 30b to introduce the heated dry air HA into the drying chamber 16 through the vents 16d and circulate the heated dry air HA in the drying chamber 16. The microprocessor 24 controls operation of the heater 28 and the fan 30 to provide a desired flow of the heated dry air HA into the drying chamber 16. The fan 30 preferably uses a magnetic bearing assembly, available from Sunon (www.sunon.com) under the name “MagLev Motor Fan” instead of ball bearing types of fan motors. This assembly allows the unit 10 to operate with less motor noise, as well as extended life, and a higher level of reliability.

The heated dry air HA may exit the drying chamber 16 via the outlet vents 16c in the lid 16a. It is believed that operation of the unit 10 to provide a desirably controlled temperature in the drying chamber 16 in combination with a desired flow of moving air breaks the surface tension bond that water molecules have on the hearing aids. The heated dry air HA air circulates throughout the drying chamber 16 and exits the drying chamber 16 through the outlet vents 16c, thereby removing moisture from the drying chamber 16.

The thermostat 24a is configured to measure the air temperature in the drying chamber 16. The thermostat 24a is preferably an integrated digital thermostat that controls the temperature within the drying chamber 16 to a high level of thermal accuracy. The microprocessor 24 is programmed to maintain the air temperature in the drying chamber 16 above a predetermined drying temperature and below a predetermined upper temperature corresponding to a temperature above which damage would occur to rechargeable batteries of the electronic devices, such as the hearing aids 12, in the drying chamber 16.

The microprocessor 24 is preferably a customizable digitally programmable microprocessor with non-volatile memory. The microprocessor 24 controls the on/off times of the light sources 20 and then the heater 28 and the fan 30 to provide a desired treatment cycle, and automatically turns off after the treatment cycle has completed or if the lid 16a has been opened. The hearing aids 12 are recharged during the treatment cycle and may be left on the charger 14 following the treatment cycle if desired. In this regard, a treatment cycle is understood to include sanitization via the light sources 20 followed by drying via the heated dry air HA.

During use of the unit 10, as depicted in FIG. 4, a user may place hearing aids attached to their manufacturer-supplied charger inside the drying chamber 16, shut the lid 16a, and press the on/off switch 22b. As shown in FIG. 5, the microprocessor 24 then initiates a sanitization cycle by turning on the light sources 20 to disinfect all exposed surfaces of the hearing aids 12. After a pre-determined time has elapsed, the microprocessor 24 turns off the light sources 20 and initiates a drying cycle by turning on the heater 28 and the fan 30.

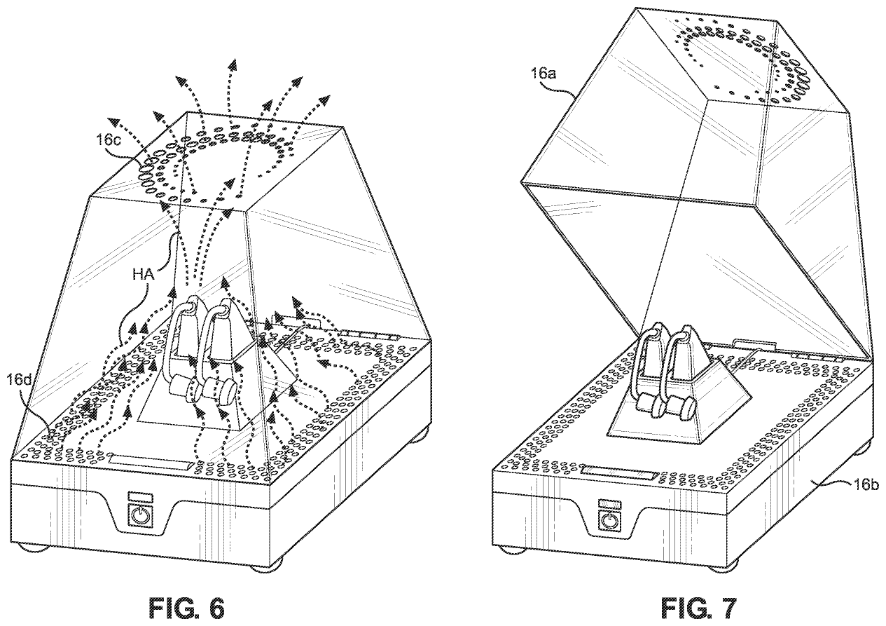

Next, as shown in FIG. 6, the heated dry air HA is introduced to the drying chamber 16 through the vents 16d of the base 16b, and circulates within the drying chamber 16, and leaves the drying chamber through the air vents 16c in the lid 16a. The circulating heated dry air HA functions to break the surface tension of water molecules on the surface of the hearing aids and draws the moisture away therefrom and out of the drying chamber 16 through the vents 16c in the lid 16a. Meanwhile, the microprocessor 24 monitors the temperature in the drying chamber using the thermostat 24a and maintains a pre-programmed temperature by controlling the output of the heater 28 and the speed of the one or more fans 30. Precise control of the temperature of the circulating air is imperative to prevent overheating of the batteries of the hearing aids and the charger. At the conclusion of the drying phase of the treatment cycle, the microprocessor 24 turns off the heater 28 and the fan 30 and the hearing aids 12 may be removed from the drying chamber 16 as shown in FIG. 7.

To accommodate electronic devices that recharge using wireless nearfield proximity charging, an embodiment of the unit 10 includes an inductive coil in the base and associated circuitry to provide the wireless nearfield proximity charging. In this embodiment, the rechargeable electronic devices may be set directly on the base above the inductive coil for charging.

### Second Embodiment

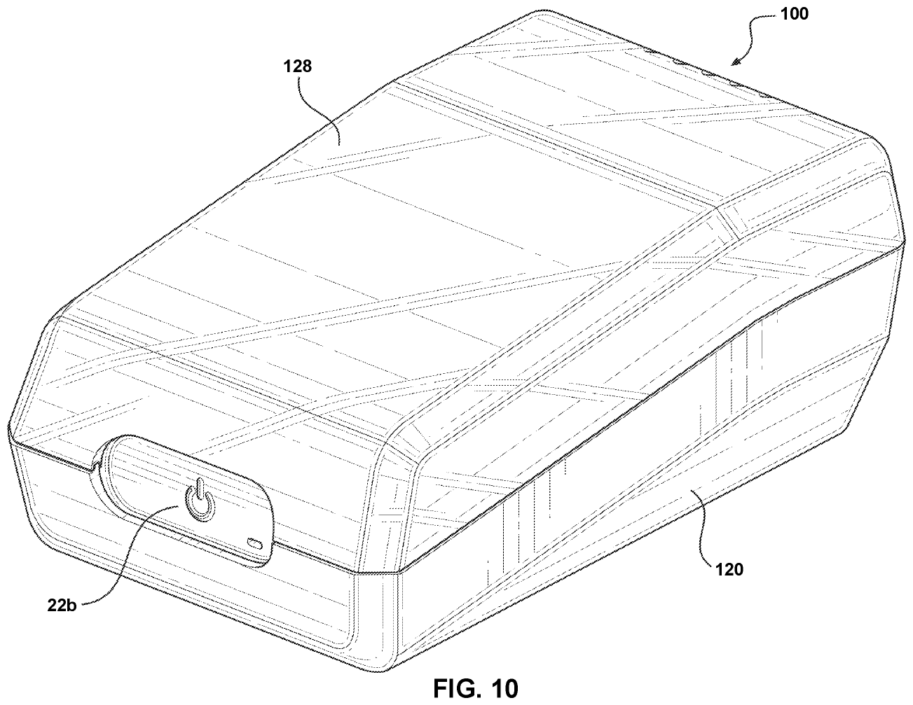

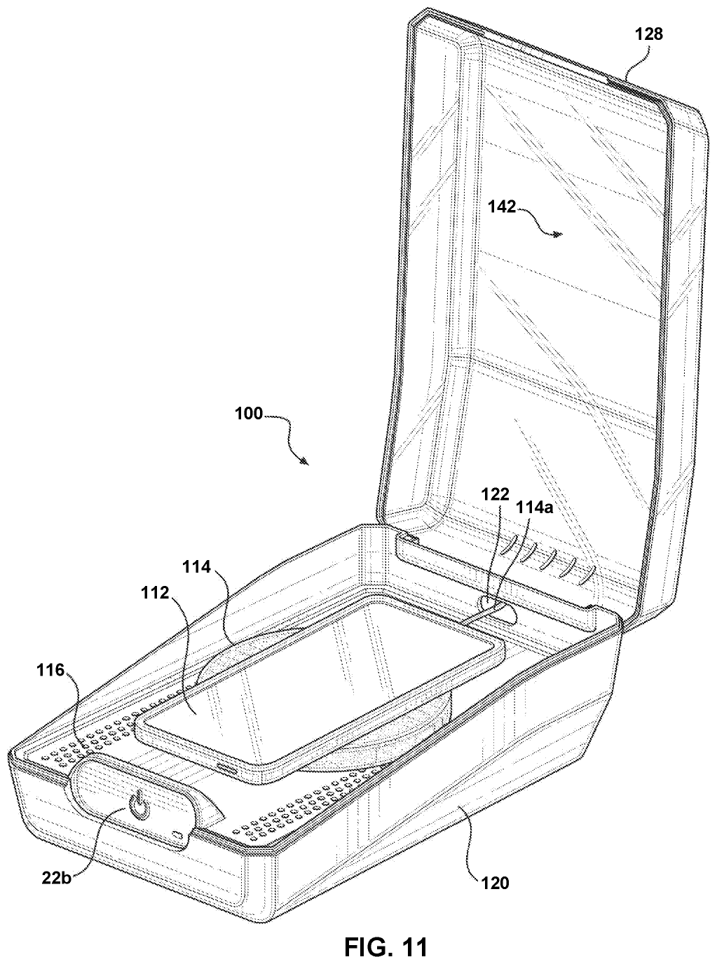

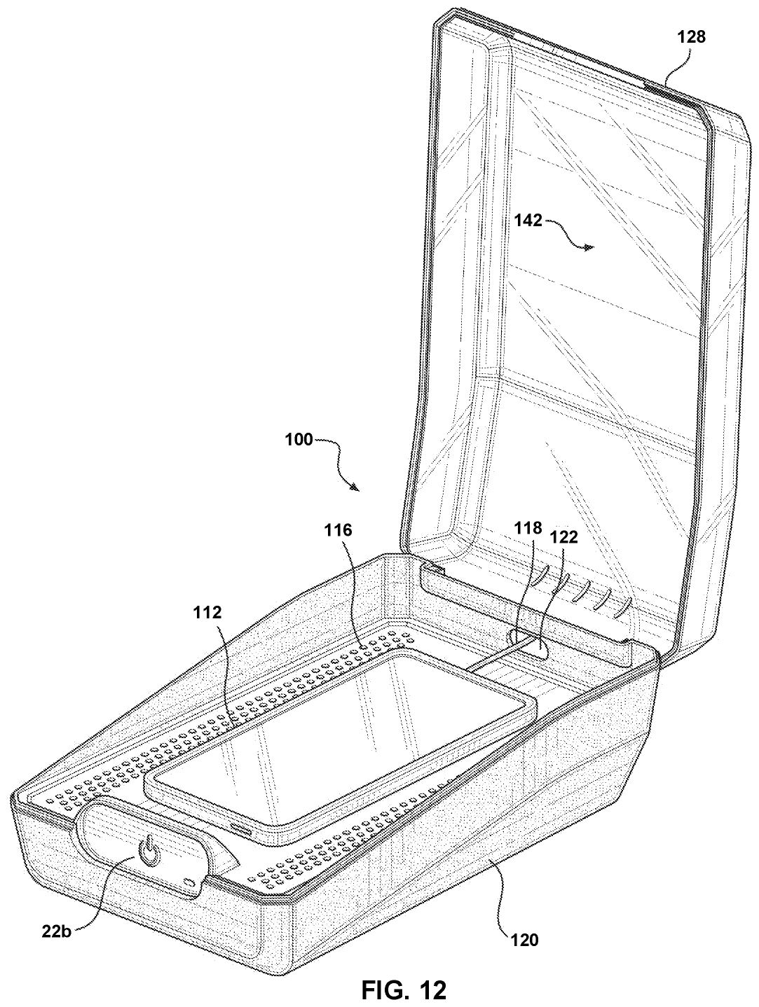

With initial reference to FIGS. 10-13, a second embodiment of a dryer and sanitizer unit 100 is depicted according to the disclosure, configured for drying and sanitizing portable rechargeable electronic devices 112 such as cell phones, hearing aids, watches, headphones, and earphones. As shown in FIG. 11, the rechargeable electronic device 112 may be a cell phone 112 disposed on a nearfield proximity charger 114 having an electric cord 114a. As shown in FIG. 12, the cell phone 112 may be placed on a base 116 of the drying and sanitizing unit 100 rather than on the charger 114. In this case, a charging cable 118 is directly connected to the cell phone 112 rather than to the proximity charger 114.

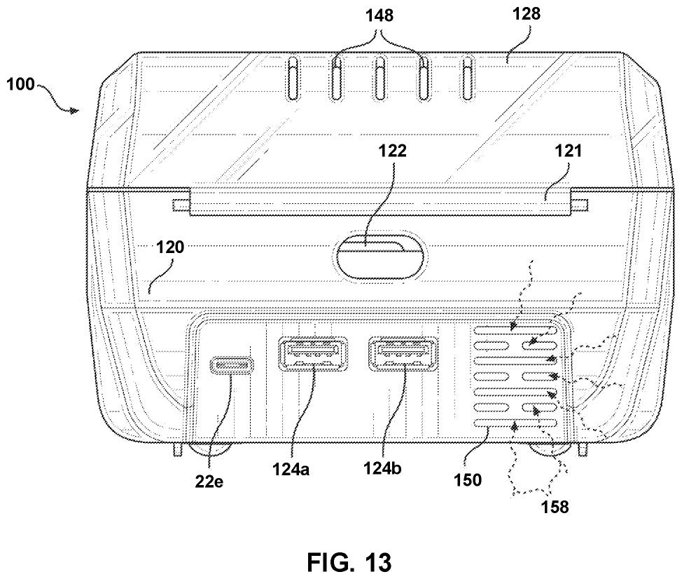

Accordingly, the dryer and sanitizing unit 100 includes a housing 120 for the base 116 having an aperture 122 therein for the cables 114a and 118. Cables 114a and 118 may be standard USB cables for connection to USB ports 124a and 124b on the housing 120 (FIG. 13). The USB ports 124a and 124b may be Type-A USB ports that enable the charging of two different products. The USB ports 124a and 124b may provide 5 to 15 watts of power to the charger and/or rechargeable electronic device. Power may be input to the dryer and sanitizer unit 100 by means of the power input 22e. Power may be provided by a standard electrical plug, a USB micro plug or USB-C plug input connected to a power source, or a solar energy input.

In this regard, initially the rechargeable electronic devices 112 are disinfected by application of sanitizing illumination. Following or simultaneous with the sanitization, heated dry air is flowed over the rechargeable electronic devices to remove moisture. The sanitization and drying operations are described in more detail hereinafter.

A lid 128 is connected to the housing 120 by a hinge 121 to provide a closed chamber between the lid 128 and the housing 120 as shown in FIG. 10. As in the first embodiment, the capacitive touch switch 22b on the housing 120 is used to activate the dryer and sanitizer unit 100. The switch 22b may be operated to provide sanitization by touching the switch once, and to provide sanitization and drying by touching the switch twice. Touching the switch three times may be used to activate only the drying system.

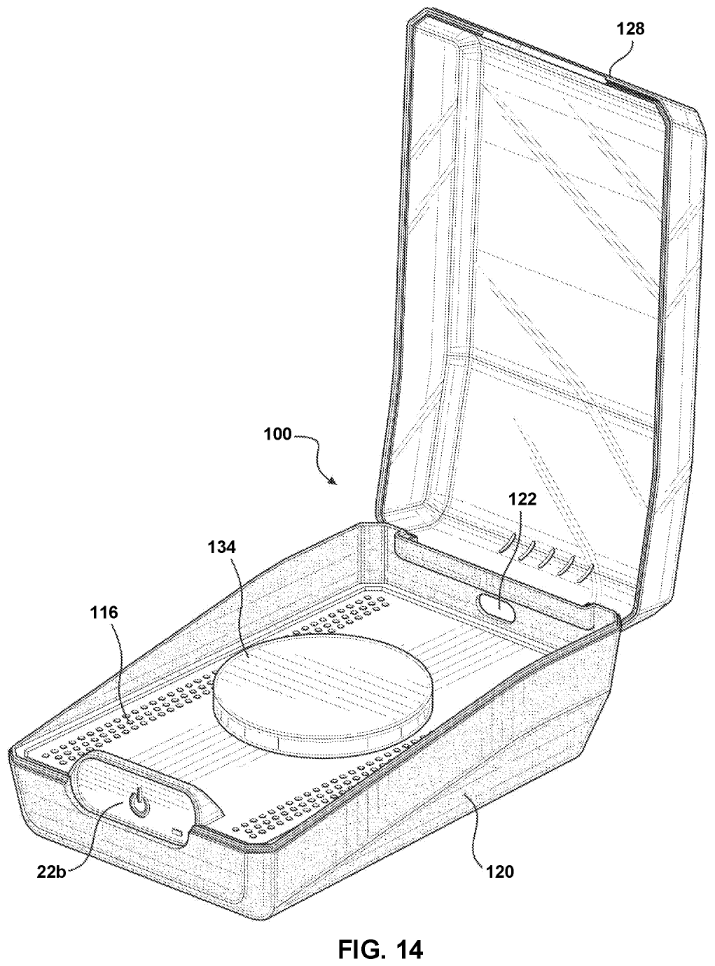

FIG. 14 illustrates an embodiment in which the base 116 contains an inductive coil and associated circuitry below a platform 134 to provide wireless nearfield proximity charging for the electronic devices placed on base 116. In this embodiment, the rechargeable electronic devices may be set directly on the platform 134 above the inductive coil for charging.

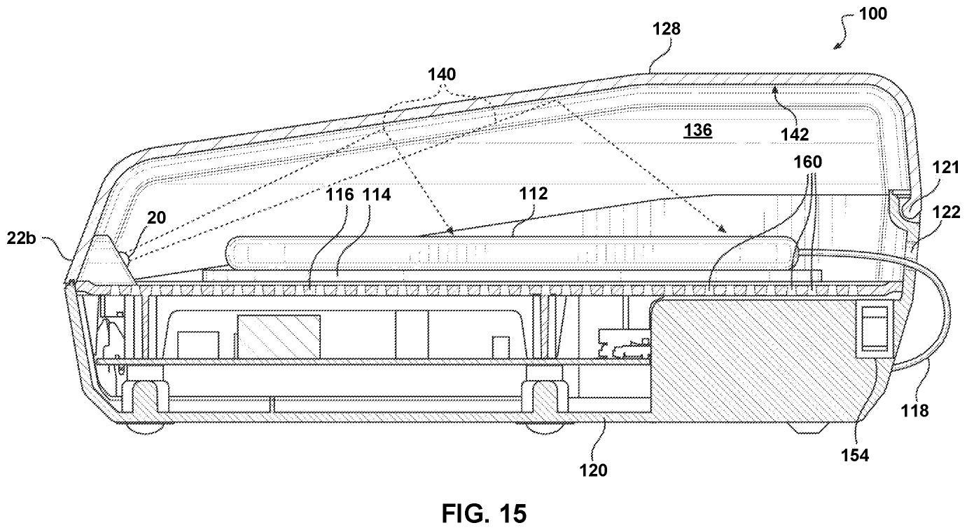

FIG. 15 is a cross-sectional view of the dryer and sanitizer unit 100 according to the second embodiment of the invention. The dryer and sanitizer unit 100 is configured to include a drying chamber 136 defined by the lid 128 over the housing 120. The lid 128 may have a variety of shapes and sizes to accommodate a variety of rechargeable electronic devices of varying size. The unit 100 also includes one or more apertures 122 for passage of electrical cords, such as the cord 118, out of the drying chamber 136. The apertures 122 are desirably located on housing 120 but could be provided on the lid 128 if desired.

The unit 100 also includes one or more disinfecting light sources 20 located within the drying chamber 136. As in the first embodiment, the disinfecting light sources 20 are preferably UV-C lamps, such as high-intensity 50 mm linear germicidal lamps operating at a wavelength of 253.7 nm and rated at 70 uW/cm². In some embodiments, the disinfecting light sources 20 may be disposed in the drying chamber 136 at an angle with respect to the inside surface 142 of the lid 128. This ensures that the disinfecting light is reflected from the inside surface 142 as illustrated by lines 140, so that the entire surface of the cell phone 112 or other rechargeable electronic device is thoroughly illuminated by disinfecting light. Accordingly, the interior planar reflective surface 142 of the lid 128 may contain a mirror, reflective metal foil, or other UV-reflective coating to reflect light from the disinfecting light sources 20 toward exposed surfaces of the rechargeable electronic devices.

The lid 128 preferably has a generally rectangular prism shape having multiple angled surfaces 142 as shown to reflect light from the disinfecting light sources 20 at multiple angles of incidence toward the rechargeable electronic devices 112.

As with the first embodiment, the heated dry air is preferably supplied at a temperature that fits the specification of the manufacturer of the batteries for the portable electronic devices 112. The flow rate and flow time of the heated dry air is selected based on observation of parameters that provide optimal drying, and the microprocessor 24 is programmed to provide a desired air temperature, time and fan speed to provide the desired drying effect.

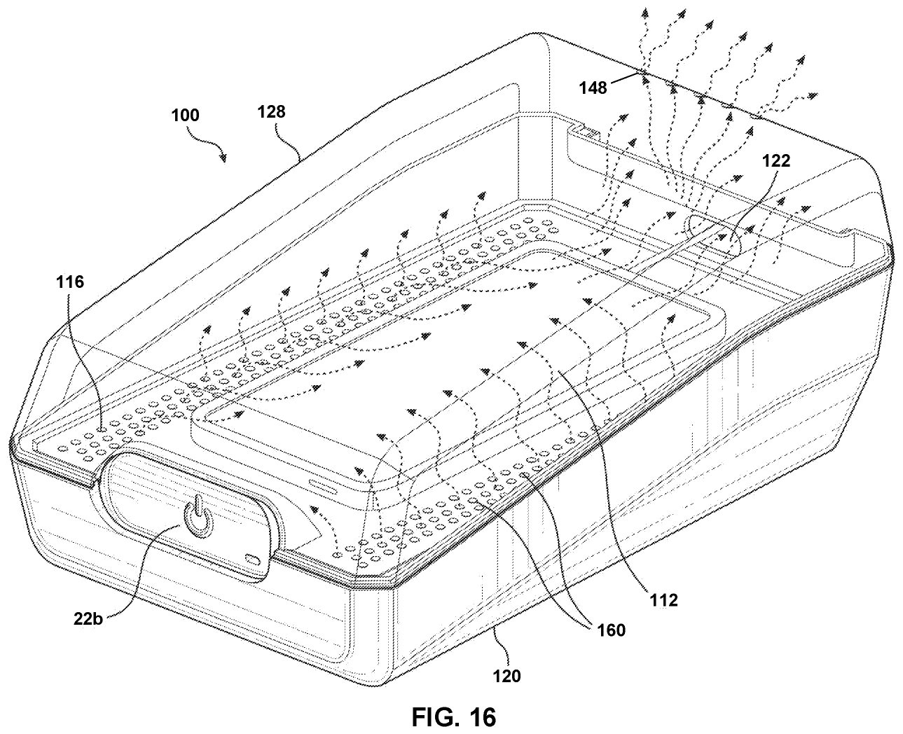

As shown in FIG. 13, the housing 120 includes air inlet vents 150 through which air enters the housing 120 to be heated so as to provide heated dry air into the drying chamber 136. As illustrated in FIG. 16, the heated dry air may exit the drying chamber 136 via outlet vents 148 in the lid 128. It is believed that operation of the unit 100 in a manner to provide a desirably controlled temperature in the drying chamber 136 in combination with a desired flow of moving air breaks the surface tension bond that water molecules have on surfaces of the rechargeable electronic devices. The heated dry air circulates throughout the drying chamber 136 and exits the drying chamber 136 through the outlet vents 148 serving to remove moisture from the drying chamber 136.

During use of the unit 100, as depicted in FIGS. 15 and 16, a user may place a cell phone 112 inside the drying chamber 136, shut the lid 128, and press the on/off switch 22b. The microprocessor 24 then initiates a sanitization cycle by turning on the light sources 20 to disinfect all exposed surfaces of the cell phone 112. After a predetermined time has elapsed, the microprocessor 24 turns off the light sources 20 and initiates a drying cycle by turning on the heater 28 and the fan 30.

As shown in FIGS. 13 and 16, an air flow (indicated by arrows 158) is introduced to the drying chamber 36 through the vents 150 of the housing 120, circulates within the drying chamber 136 through a plurality of holes 160 in the base 116, and exits the drying chamber 136 through the air outlet vents 148 in the lid 128. The circulating air is heated by the heater 28 to provide heated dry air that breaks the surface tension of water molecules on the surface of the rechargeable electronic device and draws the moisture away therefrom and out of the drying chamber 136 through the outlet vents 148 in the lid 128. Meanwhile, the microprocessor 24 monitors the temperature in the drying chamber using the thermostat 24a and maintains a pre-programmed temperature by controlling the output of the heater 28 and the speed of the fan 30. Precise control of the temperature of the circulating air is imperative to prevent overheating of the batteries of the cell phone 112. At the conclusion of the drying phase of the treatment cycle, the microprocessor 24 turns off the heater 28 and the fan 30, and the cell phone 112 may be removed from the drying chamber 136.

The entire base 116, housing 120 and lid 128 may be configured of various colors, shapes, and dimensions depending on the device(s) being charged. The materials for the base, lid and housing may be made from a wide variety of metal and plastic materials in in one embodiment may be made of flame retardant acrylonitrile butadiene styrene materials.

The foregoing description of preferred embodiments for this disclosure has been presented for purposes of illustration and description. It is not intended to be exhaustive or to limit the disclosure to the precise form disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

1. A dryer and sanitizer unit comprising:
a drying chamber for enclosing one or more portable rechargeable electronic devices, the drying chamber defined by a base and a repositionable lid that covers the base, wherein the base comprises:
a substantially horizontal surface upon which the one or more portable rechargeable electronic devices are disposed; and
air vents disposed around a perimeter of the horizontal surface through which warm air may flow into the drying chamber to contact the one or more portable rechargeable electronic devices, wherein the air vents at least partially surround the one or more rechargeable electronic devices disposed on the base; and
one or more disinfecting light sources disposed within the drying chamber.

2. The dryer and sanitizer unit of claim 1, wherein the lid includes one or more passages through which electrical cords associated with the one or more portable rechargeable electronic devices may pass out of the drying chamber.

3. The dryer and sanitizer unit of claim 1, wherein the base includes one or more passages through which electrical cords associated with the one or more portable rechargeable electronic devices may pass out of the drying chamber.

4. The dryer and sanitizer unit of claim 1, further comprising a thermostat operably associated with the drying chamber for measuring air temperature in the drying chamber.

5. The dryer and sanitizer unit of claim 4, further comprising a microprocessor operably associated with the thermostat for controlling the air temperature in the drying chamber to maintain the air temperature above a predetermined drying temperature and below a predetermined upper temperature corresponding to a temperature above which rechargeable batteries of the portable rechargeable electronic devices are vulnerable to overheating.

6. The dryer and sanitizer unit of claim 1, wherein the one or more disinfecting light sources are disposed in the base.

7. The dryer and sanitizer unit of claim 1, further comprising a fan and a heater in flow communication with the air vents to cause warm air to flow through the air vents into the drying chamber.

8. The dryer and sanitizer unit of claim 7, further including air vents disposed in the lid to promote flow of the warm air over the portable rechargeable electronic devices and to provide a path for the warm air to exit the drying chamber.

9. The dryer and sanitizer unit of claim 7, wherein the disinfecting light sources are operated for a predetermined time sufficient to disinfect the portable rechargeable electronic devices, after which the disinfecting light sources are turned off and the heater and fan are operated for a predetermined drying cycle.

10. The dryer and sanitizer unit of claim 1, wherein the lid has a square pyramidal shape with a flat top.

11. The dryer and sanitizer unit of claim 1, wherein an interior surface of the lid is UV-reflective.

12. The dryer and sanitizer unit of claim 1, further comprising a lid open detection circuit operably associated with both the lid and the one or more disinfecting light sources, wherein the lid open detection circuit is operable to turn off the one or more disinfecting light sources if the lid is opened while the one or more disinfecting light sources are turned on.

13. The dryer and sanitizer unit of claim 1, further comprising a wireless nearfield proximity charger integrated into the base.

14. A dryer and sanitizer unit comprising:
a drying chamber for enclosing one or more portable rechargeable electronic devices, the drying chamber defined by a base and a repositionable lid that covers the base, wherein the base comprises:
a substantially horizontal surface upon which the one or more portable rechargeable electronic devices are disposed; and
air vents disposed around a perimeter of the horizontal surface through which warm air may flow into the drying chamber to contact the one or more portable rechargeable electronic devices, wherein the air vents at least partially surround the one or more rechargeable electronic devices disposed on the base; and
a fan in flow communication with the air vents, the fan for causing air to flow through the air vents into the drying chamber;
a thermostat operably associated with the drying chamber for measuring temperature of the air flowing into in the drying chamber;
a heater for heating the air flowing into the drying chamber; and
a microprocessor in electrical communication with the thermostat, heater, and fan, the microprocessor programmed to control one or both of the heater and the fan to maintain the temperature of the air flowing into the drying chamber above a predetermined drying temperature and below a predetermined upper temperature above which rechargeable batteries of the portable rechargeable electronic devices are vulnerable to overheating.

15. The dryer and sanitizer unit of claim 14, further comprising one or more disinfecting light sources disposed within the drying chamber.

16. The dryer and sanitizer unit of claim 14, further comprising a wireless nearfield proximity charger integrated into the base.

17. A dryer and sanitizer unit comprising:
a drying chamber for enclosing one or more portable rechargeable electronic devices, the drying chamber defined by a base and a repositionable lid that covers the base, wherein the base comprises:
a substantially horizontal surface upon which the one or more portable rechargeable electronic devices are disposed; and
air vents disposed around a perimeter of the horizontal surface through which warm air may flow into the drying chamber to contact the one or more portable rechargeable electronic devices, wherein the air vents at least partially surround the one or more rechargeable electronic devices disposed on the base; and
a wireless nearfield proximity charger integrated into the base.

18. The dryer and sanitizer unit of claim 17, further comprising one or more disinfecting light sources disposed within the drying chamber.
